(19) United States
(12) Patent Application Publication
(10) Pub. No.: US 2026/0202352 A1
(43) Pub. Date: Jul. 16, 2026

(54) DEVICE, SYSTEM, AND METHOD FOR OIL-CONTAINING ELECTRICAL POWER DEVICES OR TRANSMISSION DEVICES

(71) Applicant: E.ON SE, Essen (DE)

(72) Inventors: Rajkiran KUMAR, Muenchen (DE); Darren VALENTINE, Muenchen (DE); Eugenio SCIONTI, Muenchen (DE); Florian FECHER, Muenchen (DE)

(73) Assignee: E.ON SE, Essen (DE)

(21) Appl. No.: 19/135,966

(22) PCT Filed: Sep. 1, 2023
(86) PCT No.: PCT/EP2023/073993

§ 371 Date: Jun. 5, 2025

(30) Foreign Application Priority Data
Dec. 19, 2022 (DE) 10 2022 133 900.9

(51) Int. Cl. G01N 22/00; G01N 33/28; G06F 18/241; H01F 27/12

(52) U.S. Cl. CPC G01N 22/00; G01N 33/28; G06F 18/241; H01F 27/12

(57) ABSTRACT

A device for an oil-containing electrical power device or transmission device may include a sensor configured to transmit one or more electromagnetic signals with variable frequencies in the range from 1 Hz to 3000 GHz at the same time into oil in the oil-containing electrical power device or transmission device and to receive reflected and/or propagated electromagnetic signals, and a processing device that is configured to compare the received signals in the time domain and/or frequency domain with first and second signals in the time domain and/or frequency domain, wherein the first signals in the time domain and/or frequency domain are classified with respect to a physical property of oil, the second signals in the time domain and/or frequency domain are classified with respect to a chemical property of oil, and the processing device is further configured to determine, based on the comparisons, an oil quality.

### TECHNICAL FIELD

The present disclosure relates to a device, a system and a method for oil-containing electrical power devices or transmission devices. In particular, the present disclosure relates to a device and a system for an oil-containing electrical power device or transmission device with which the oil quality in the oil-containing electrical power device or transmission device can be determined, and to a method for a plurality of oil-containing electrical power devices or transmission devices with which the oil quality in the oil-containing electrical power devices or transmission devices can be determined.

### BACKGROUND

Oil-insulated distribution transformers, also referred to as electrical oil transformers, are power transformers that are mostly used in energy distribution networks. Such oil transformers exist in various configurations.

Hermetic transformers, for example, are hermetically sealed oil transformers without an expansion tank or gas cushion, which prevents the oil from coming into contact with the atmosphere and thus avoids accelerated aging of the oil.

Another type of oil transformer has an oil expansion tank arranged above the transformer tank and connected to the transformer tank via a flow channel. By means of the oil expansion tank, changes in volume of the oil can be compensated. The oil expansion tank serves to accommodate an oil volume that arises due to the thermal expansion of the oil when temperature fluctuations occur in the transformer because of load changes or changes in the ambient temperature. Inside the oil expansion tank, there can be a compressible membrane filled with air. Depending on the degree of expansion of the oil in the transformer, the membrane is compressed, with the interior of the transformer tank, the oil expansion tank and the flow channel forming a closed system. A magnetic oil level gauge (MOG) can be used to monitor the oil level in the oil expansion tank.

DE202008017356U1 describes an oil transformer having a transformer tank filled with oil, in which the transformer core with primary and secondary windings is arranged. For insulation, the primary and secondary windings can be wrapped with a cellulose paper. The oil serves as an electrical insulation medium and as a cooling medium for dissipating the heat losses generated during transformer operation. Depending on the operating oil temperature, the oil expands and contracts in its volume.

As the oil transformer ages, the oil is contaminated by moisture and fiber materials in the insulating material of the windings. In addition, dissolved gases that arise through chemical reactions in the oil can contaminate the oil. In order to ensure safe operation and to avoid interruptions in operation or power failures, the oil must be checked at regular intervals and replaced if necessary.

Monitoring of the oil is usually carried out in one of the following ways: An oil sample is manually taken from the transformer and sent to a laboratory for analysis. Tests of the insulation resistance and the dielectric breakdown voltage of the oil then take place in the laboratory. A furan analysis can also be carried out in the laboratory. Alternatively, it is known to analyze the oil at regular intervals in a measuring device. In this way, gas chromatography can be performed, which, however, has the disadvantage of being time-consuming and costly and requiring an expert to carry it out. Furthermore, photoacoustic spectroscopy can be carried out.

Known techniques for monitoring the oil in an oil transformer also have the following disadvantages: some techniques cannot be retrofitted and require a relatively high personnel expenditure for installing and configuring the measuring devices. Often, it is also necessary for the transformer not to be in operation and to be switched off during installation of the measuring devices. Furthermore, known measuring techniques can be influenced by electromagnetic impulses. For example, measuring sensors that are directly attached to the surface of the main tank of the transformer can be influenced by partial discharges. Such partial discharges can generate electromagnetic impulses in the ultra-high frequency range (300 MHz to 3 GHz), which can lead to measurement errors or measurement inaccuracies in the measuring devices. The ambient and/or surface temperature of the transformer can also cause problems. Electronic measuring devices that are operated, for example, near the limb and yoke area of the transformer are exposed to high temperatures (temperature rise of the oil due to high-voltage loads). The increased temperature can cause the measuring devices to malfunction, leading to measurement errors or inaccuracies, up to failures of the measuring devices. Measuring devices that collect oil samples from a drain valve at the bottom of the oil transformer's main tank can also be mixed with contaminated particles. Fibers and moisture from the insulation materials can combine with the oil and lead to residues settling at the bottom of the transformer. The oil samples taken in this area are often contaminated by residues, which can lead to an inaccurate analysis of the oil quality.

### SHORT SUMMARY

The present disclosure is based on the object of providing a device, a system and a method for oil-containing electrical power devices or transmission devices, with the aid of which the oil quality in an oil-containing electrical power device or transmission device can be determined in a simple and precise manner.

To achieve this object, a device, in particular a measuring device, is proposed for an oil-containing electrical power device or transmission device, which comprises the following: a sensor that is configured to transmit one or more electromagnetic signals with variable frequencies in the range from 1 Hz to 3000 GHz, in particular with variable frequencies in the range from 300 MHz to 300 GHz, at the same time into the oil in the oil-containing electrical power device or transmission device and to receive reflected and/or propagated electromagnetic signals, and a processing device that is configured to compare the received signals in the time domain and/or frequency domain with first and second signals in the time domain and/or frequency domain, wherein the first signals in the time domain and/or frequency domain are classified with respect to a physical property of oil, the second signals in the time domain and/or frequency domain are classified with respect to a chemical property of oil, and the processing device is further configured to determine, based on the comparisons, an oil quality of the oil in the oil-containing electrical power device or transmission device.

The oil-containing electrical power device or transmission device can be any type of oil-containing electrical power device or transmission device that uses oil for insulation, cooling or normal operation. A preferred embodiment of the oil-containing electrical power device or transmission device is an oil transformer. The following disclosure and the exemplary embodiments refer to oil transformers. It should be noted, however, that whenever an oil transformer is disclosed below, an oil-containing electrical power device or transmission device is also meant and can accordingly be replaced by an oil-containing electrical power device or an oil-containing electrical transmission device.

The oil transformer can be any type of oil transformer, in particular an oil transformer with an oil expansion tank or an oil transformer without an oil expansion tank. The sensor can, for example, comprise a baseband transmitter, a baseband receiver and a digital backend, with the aid of which the oil quality in the oil transformer can be determined by means of the electromagnetic signals generated by the baseband transmitter and received by the baseband receiver. The oil quality can be one of the quality features defined in IEC 60422. The sensor can thus be a contactless near-field sensor for dielectric spectroscopy. The sensor can also be configured to perform broadband dielectric spectroscopy (BDS) or electrochemical impedance spectroscopy. If the sensor, for example, is configured for ultra-wideband impedance spectroscopy, the oil can be subjected to a pulsed alternating current signal. Furthermore, a combined frequency domain/time domain technique can be used for characterizing the oil. In order to improve detection accuracy, the sensor can generate a baseband signal that is produced by combining several upconverted Gaussian signals. Additional components, such as amplifiers and ADC (analog-digital) or DAC (digital-analog) semiconductor electronics, can be provided in the sensor.

The sensor is configured to generate electromagnetic signals with a frequency of 1 Hz to 3000 GHz and to transmit them to an antenna which sends the electromagnetic signals into the oil. Electromagnetic signals with a frequency of 1 Hz to 3000 GHz are then received by the sensor via the antenna. The antenna can be designed as a single unit with a transmitting and receiving antenna (in this case referred to as a sensor module), so that signals sent into the oil are reflected/propagated and received again by the antenna. In particular, it can be a transceiver antenna. The sensor can also be separate from the antenna. In this arrangement, the antenna transmits electromagnetic signals through the oil, and the propagating signals are received by the sensor at a location different from the antenna. The signals can, in particular, be pulsed signals in the picosecond range. The interactions of electromagnetic waves with frequencies ranging from 1 Hz to 3000 GHz have the advantage that they generally do not pose serious health risks to humans and still provide good measurement results.

After propagating through the oil, the transmitted electromagnetic signal becomes deformed, which is why the received electromagnetic signal has a different phase angle and a different frequency than the originally transmitted signal. By convolving the received deformed signal with the ideal signal (original signal), an impulse response of a certain shape is obtained. The impulse response is then sent to an analog-to-digital converter (ADC) to perform a fast Fourier transform (FFT). The FFT is then examined in detail and compared.

The frequency of the signals can be adjusted and/or selected depending on the type and level of detail of the assessment information needed. For a more detailed assessment of the oil quality, a suitable frequency (either in the lower or upper frequency range) can be chosen. Even better measurement results can be obtained if the sensor is configured to generate, transmit and receive electromagnetic signals with a frequency of 300 MHz to 300 GHz. In particular, the sensor can be configured to be operated in the ultra-wideband (UWB) range. The UWB range from 0.1 GHz to 6 GHz allows for revealing (very detailed) studies on the behavior of oil when interacting with electromagnetic waves. The sensor can also be configured to measure a temperature, vibration and/or gas generation of the oil.

The processing device can be a computing device, such as a laptop or tablet computer. The processing device can be configured to apply a fast Fourier transform to the received signals. The processing device can be in communication with the sensor via a data cable, the Internet, a wireless network or a cellular network. The processing device and the sensor can also be integrated into one unit. The antenna, the processing device and the sensor can also be integrated into one unit. Furthermore, the processing device can be a cloud server.

For the comparisons carried out in the processing device, amplitude values (e.g., in dB) over time (e.g., in ms) can be compared with predefined and classified amplitude values (e.g., in dB) over time (e.g., in ms). In addition, a power spectrum calculated by means of a fast Fourier transform can be used, i.e., amplitude values (e.g., in dB) over frequency (e.g., in GHz) can be compared with predefined and classified amplitude values (e.g., in dB) over frequency (e.g., in GHz). The comparisons can be carried out using correlations (digital signal processing) or any type of output in different scales (e.g., logarithmic scale) and/or different graphical representations (e.g., Nyquist plot, Bode plot, etc.) in order to compare changes in phase angle and amplitude. In the comparisons, either signals in the time domain or signals in the frequency domain can be compared in each instance.

Thus, signals in the time domain and/or frequency domain that are classified with respect to a physical property of oil and a chemical property of oil, respectively, are used to determine an oil quality of the oil in the oil transformer, thereby enabling a simple and precise determination of the oil quality in the oil transformer.

The physical property can be one of viscosity, flash point, interfacial tension, color and density, and classification with respect to the physical property of oil concerns an oil quality. The chemical property can be one of acid value, chemical composition, in particular changes in the chemical composition that arise through involvement of dissolved gases, moisture and the influence of paper polymerization, and classification with respect to the chemical property of oil concerns an oil quality. These physical and chemical properties can enable an even more precise determination of the oil quality in the oil transformer.

In order to further improve the determination of the oil quality in the oil transformer, the processing device can be configured to compare the received signals in the time domain and/or frequency domain with third and fourth signals in the time domain and/or frequency domain, wherein the third signals in the time domain and/or frequency domain are classified with respect to a physical property of oil that differs from the classified physical property of oil of the first signals in the time domain and/or frequency domain, and the fourth signals in the time domain and/or frequency domain are classified with respect to a chemical property of oil that differs from the classified chemical property of oil of the second signals in the time domain and/or frequency domain. As with the first signals, the physical property in the third signals can be one of viscosity, flash point, interfacial tension, color and density, and classification with respect to the physical property of oil concerns an oil quality. Correspondingly, as with the second signals, the chemical property in the fourth signals can be one of acid value, chemical composition, in particular changes in the chemical composition that arise through involvement of dissolved gases, moisture and the influence of paper polymerization, and classification with respect to the chemical property of oil concerns an oil quality.

The processing device can also be configured to determine, based on the transmitted and received signals, an impedance (Z), a conductance (G), an admittance (Y), a susceptance (B) and/or combinations thereof in the time domain and/or frequency domain, and to compare the impedance, the conductance, the admittance, the susceptance and/or the combinations thereof in the time domain and/or frequency domain with the first and second signals in the time domain and/or frequency domain. For a more precise determination of the oil quality in the oil transformer, the impedance, the conductance, the admittance and/or the susceptance in the time domain and/or frequency domain can additionally be compared with the third and fourth signals in the time domain and/or frequency domain. In this way, an impedance value of the oil is determined, with the impedance (Z) being a combination of the specific resistance, the dielectric constant and the permeability (which can be considered as 1, as oil is not magnetic).

The transmitted signal (the transmitted signal can be either a single signal or multiple signals with different frequencies that are transmitted at the same time) can be any efficient multi-frequency signal (sine, cosine, etc.) for determining the electrical parameters Z, G, Y and/or B of the oil.

The larger the measurement frequency range, the more detailed the differences in the specific frequency range become. The proposed sensor technology is therefore able to measure the electrical parameters of oil over a wide frequency range, for example in an ultra-wideband range from 0.1 to 6 GHz, which provides very detailed information about the behavior of oil when interacting with electromagnetic waves.

The device can include a machine learning module that is configured to determine, based on the impedance, the conductance, the admittance, the susceptance and/or the combinations thereof, values relating to the physical property and the chemical property of the oil. The values can then be interpreted and related to a performance and/or assessments of the oil transformer.

The device can also include a machine learning module that is configured to optimize processing of machine learning models and the comparisons in the processing device, based on the classifications of the first and second signals, with the machine learning models using the first and second signals as training data. The machine learning models can also be configured to optimize the comparisons in the processing device, based on the classifications of the third and fourth signals, with the machine learning module using the third and fourth signals as training data.

Furthermore, machine learning models can be provided that are configured to be processed on a machine learning module and to determine, based on the impedance, the conductance, the admittance, the susceptance and/or the combinations thereof, values relating to the physical property and the chemical property of the oil.

Using collected data stored on a cloud platform, additional machine learning models can be developed. These machine learning models find patterns and structures in the data coming from one or more sensors. The trained machine learning models are sent to the processing device in order to enable a calculation/prediction/classification of the data (real-time data) from the sensor. Thus, the machine learning models can comprise a variety of machine learning models. The machine learning module consists of the necessary memory and computing hardware, e.g. CPU, GPU, NPU, in order to make predictions from machine learning models as well as to enable the training and development of machine learning models.

The machine learning models can also learn based on regressions. All machine learning models can be trained and used for inference in the device as part of the machine learning module, or they can be trained and used for inference on another computer such as a laptop or a cloud computer. The machine learning module can be located in the device or on a cloud platform. The machine learning module can, for example, be hosted or developed on the Microsoft Azure platform. In this case, the processing device can communicate with the machine learning module via the Internet. The processing device and the machine learning module can also both be hosted on a cloud platform, such as the Microsoft Azure platform. In this case, the antenna communicates with the cloud platform via an additional communication device. The machine learning module can be provided locally in the device. The data can also be sent from an aggregation device via a highly secure gateway to the cloud level. Applications can be operated at the cloud level with this data, which are based on the Microsoft Azure platform or the Amazon Cloud platform.

The machine learning models take as inputs the signals recorded by the sensor. The machine learning models have been trained in such a way that they map this input to a physical or chemical characteristic that is not measured directly by the sensor. The outputs of the machine learning models are either a predicted value or a probability distribution for a range of values. The output can also be an artificial metric created to represent the overall condition of the transformer or the expected performance under certain circumstances.

Mathematical functions with different weights and parameters are used to associate inputs with outputs. These weights and parameters are determined from sensor signal data recorded from transformer oil with a known condition, e.g. from the laboratory. Finding the weights and parameters is referred to as training and can be done with a gradient descent algorithm. The training process for the machine learning model can be carried out in the cloud or on a device in an edge/fog layer. The inference or the creation of predictions, that is, the conversion of inputs into outputs without changing the parameters or weights, can also be carried out on the device in an IoT layer, in the edge/fog layer or on a cloud computer/system.

Once sufficient data is stored in a cloud database, unsupervised machine learning models can be developed. Since data is recorded at regular intervals relatively quickly, the amount of data from the sensor stored in the cloud is larger than the amount of laboratory data/known oil condition data. The data from a sensor stored in the cloud is unlabeled, meaning the actual oil quality and condition of the oil transformer are not known. By processing the data from a single sensor or multiple sensors, which are installed on one or more oil transformers, stored in the cloud, patterns or structures in the data can be recognized that provide information about the relative changes in the oil or the oil transformer conditions.

The identified patterns and structures in the data can be used for qualitative and quantitative monitoring of the oil transformer and can be further utilized in applications such as lifetime prediction of the oil transformer, prediction of transformer performance and capacity detection based on oil quality, active load management based on transformer capacity and transformer recommendations.

The data used for training is stored in a database in the cloud, and if the training process is carried out on a cloud device, the learned weights and parameters are sent to the inference device (in the cloud, IoT or edge layer).

The sensor can be configured to transmit the electromagnetic signals at or in various openings or valves of the oil-containing electrical power device or transmission device, in particular of the oil transformer. Subsequent measurements can, for example, be carried out at various valve and/or drain openings of the oil transformer, which are located at the top, bottom or side of the oil transformer. Measurements can also be made at a valve between the radiator and the main tank of the oil transformer. For an opening located at the bottom of the oil transformer, a pump is not needed for oil extraction. Other means of making the oil available to the sensor can be provided.

The sensor can also comprise a plurality of sensors at or in various openings of the oil-containing electrical power device or transmission device, whereby the plurality of sensors is configured to transmit one or more electromagnetic signals with variable frequencies in the range from 1 Hz to 3000 GHz, in particular with variable frequencies in the range from 300 MHz to 300 GHz, at the same time into the oil and to receive reflected electromagnetic signals, and to process the received signals.

The device can further comprise: an aggregation device with a communication device that is configured to receive the processed signals from the plurality of sensors, with the aggregation device being configured to aggregate the processed signals from the plurality of sensors, and the communication device being configured to transmit the aggregated signals. The aggregation device can also be configured to aggregate the signals received from the plurality of sensors and comprise a communication device that is configured to transmit the aggregated signals. The aggregation device can be arranged in the sensor or in the processing device. Accordingly, the communication device can be arranged in the sensor or in the processing device. If the aggregation device and the communication device are arranged in the processing device, the communication device transmits the aggregated signals to a central unit. The central unit can be, for example, a cloud platform.

The communication device can also be configured to communicate with SCADA systems (Supervisory Control and Data Acquisition), either via intelligent electronic devices (IEDs) or directly with remote telemetry units (RTUs) using the standard IEC 61850 client-server protocol (e.g. XMPP open-source protocol based on IEC 61850 or IEC 61850 MMS protocol).

The processing device can also be configured to process the received signals at the same time.

The device can further comprise a temperature stabilizer that is configured to keep the temperature of the oil constant while transmitting and receiving the electromagnetic signals. This can further improve the determination of the oil quality of the oil transformer.

The device can furthermore comprise an automated and controllable calibration device that is configured to remove irregularities in the received electromagnetic signals. This can also further improve the determination of the oil quality of the oil transformer.

The device can further comprise an antenna that is configured to transmit the electromagnetic signals generated by the sensor into the oil and to receive the reflected and/or propagated electromagnetic signals, wherein the antenna comprises a complementary split-ring resonator, planar resonance-based sensor electrodes or electrodes/probes that are designed to optimize parasitic effects and double-layer effects of the oil. The antenna is electrically connected to the sensor. In order to avoid measurement inaccuracies, the antenna can be arranged or mounted directly on the oil expansion tank.

The sensor can be located away from the oil expansion tank, for example more than 1 m away. The antenna and the sensor can thus be connected via cables or via radio interfaces (for example via WLAN or Bluetooth). The antenna can be a transmitting antenna and a receiving antenna, an integrated transmitting and receiving antenna, or two integrated transmitting and receiving antennas. In particular, the antenna(s) can be designed as Vivaldi antennas. Vivaldi antennas can be used advantageously because of their large bandwidth, low cross-polarization and constant group delay. The antenna(s) can also be designed as ultra-wideband (UWB) antennas, spiral antennas or other types of antennas. The antenna and the sensor can also be designed as a single unit or module, for example in one housing. Furthermore, the sensor can comprise a communication interface for communicating with a cloud computer. The sensor can also be retrofitted according to a mechanical design that is compatible with the valves of the oil transformer.

In order to attach the antenna, a cell and means for conveying oil from the oil transformer to the cell, a fastening device can be provided that enables a detachable attachment of the antenna, the cell and the means for conveying oil from the oil transformer to the cell on or in the oil transformer. Other attachment forms, such as screws or a magnetic attachment, are also conceivable. The more extensive the sample data is, the better the analysis becomes. Therefore, the device can be attached in different places of an oil transformer (top valve, bottom valve, drain valve, radiator valve, openings on the oil expansion tank) in order to obtain various measurement results. Placing the sensors in different positions (top, bottom, at the drain, at the radiator valve) is beneficial for the overall analysis, for example, comparing the lower oil temperature with the upper oil temperature is a good method to check whether the insulating fluid circulates properly. A lack of circulation leads to accelerated deterioration of the transformer insulation system.

The processing device can also be configured to make lifetime predictions for components of the oil-containing electrical power device or transmission device based on the determined oil quality, to detect anomalies of the oil-containing electrical power device or transmission device, to make predictions of transformer performance and capacity detection of the oil-containing electrical power device or transmission device, to perform active load management based on a transformer capacity of the oil-containing electrical power device or transmission device, and/or to make transformer recommendations of the oil-containing electrical power device or transmission device from a plurality of oil transformers. Furthermore, recommendations for oil transformers can be made based on historical environmental, geographical and transformer performance data in relation to the oil quality.

The device can also comprise a cloud platform that is configured to make lifetime predictions for components of the oil-containing electrical power device or transmission device based on the determined oil quality, to detect anomalies of the oil-containing electrical power device or transmission device, to make predictions of transformer performance and capacity detection of the oil-containing electrical power device or transmission device, to perform active load management based on a transformer capacity of the oil-containing electrical power device or transmission device, and/or to make transformer recommendations of the oil-containing electrical power device or transmission device from a plurality of oil transformers. Furthermore, recommendations for oil transformers can be made based on historical environmental, geographical and transformer performance data in relation to the oil quality.

The stated object is also achieved by a system comprising one or a plurality of oil-containing electrical power devices or transmission devices and the aforementioned device for each oil-containing electrical power device or transmission device.

The stated object is further achieved by a method for determining an oil quality of an oil-containing electrical power device or transmission device with a device as described above, the method comprising the following steps: arranging a plurality of sensors of the device at or in different openings and/or valves of the oil-containing electrical power device or transmission device, and transmitting, by the plurality of sensors, the electromagnetic signals into the oil in the oil-containing electrical power device or transmission device. The aggregated signals can then be sent to a central unit, such as a cloud platform, for data analysis.

Taking oil samples at different locations of an oil transformer (for example, top valve, bottom valve, valves near radiators, vent openings or valves on the oil expansion tank) improves the overall results of the oil quality assessment method.

Over time, the chemical and physical properties of the oil deteriorate, and there are correlations between the physical and chemical properties of the oil and the performance of the oil transformer. For example, the interfacial tension, IFT (physical property), is inversely related to the operating time of the oil transformer, while the acid value (chemical property) is directly related to the operating time of the oil transformer, and the moisture content in the oil (chemical property) has a very strong inverse relationship to the breakdown voltage of transformer oil.

Deriving the chemical and physical properties of the oil in real time from the results of electrochemical impedance spectroscopy and linking them to the performance of the transformer help in the qualitative and quantitative real-time monitoring and maintenance of the oil.

The aspects and variants described above can be combined without this being explicitly described. Each of the described embodiments is thus optional with respect to each embodiment or combinations thereof. The present disclosure is therefore not limited to the individual embodiments and variants in the described order or a specific combination of the aspects and embodiments.

### SHORT DESCRIPTION OF THE DRAWINGS

Further advantages, details and features of the methods, devices and systems described here result from the following description of exemplary embodiments and from the figures.

FIG. 1 shows a schematic representation of a first exemplary embodiment of an oil transformer with an oil expansion tank;

FIG. 2 shows a schematic representation of the oil expansion tank of FIG. 1 with a device for determining the oil quality;

FIG. 3 shows a schematic representation of a second exemplary embodiment of an oil transformer with devices for determining the oil quality; and

FIG. 4 shows a schematic representation of a third exemplary embodiment of a system for a plurality of oil transformers.

### DETAILED DESCRIPTION

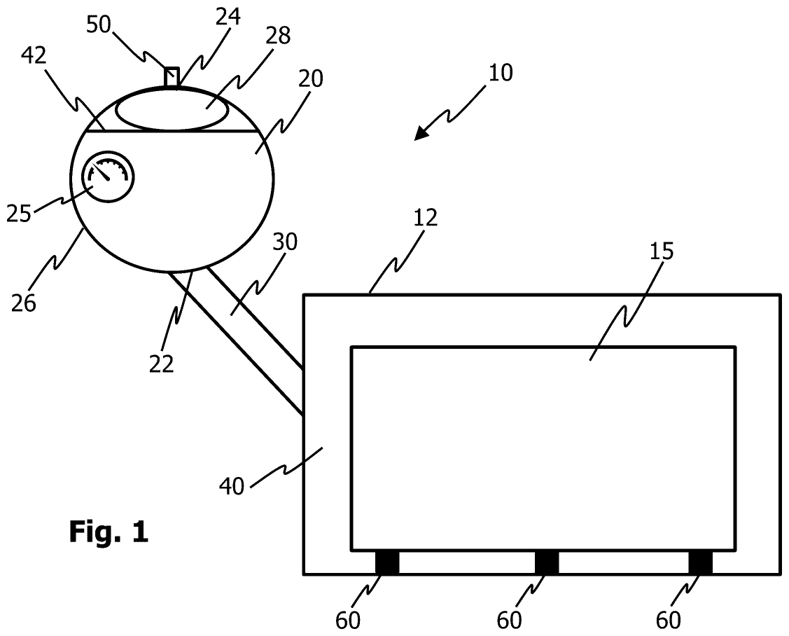

FIG. 1 shows a schematic representation of one exemplary embodiment of an oil transformer with an oil expansion tank. Oil transformer 10 comprises a transformer tank 12 and an oil expansion tank 20. A flow channel 30 connects the transformer tank 12 to a first opening 22 in the oil expansion tank 20. The first opening 22 is arranged at a lower end of the oil expansion tank 20. The transformer tank 12 includes a corresponding opening. In the transformer tank 12, the transformer 15 is mounted on support blocks 60 in an oil bath 40. The oil expansion tank 20 in FIG. 1 is shown by way of example above the transformer tank 12 in a cylindrical form. Other shapes (e.g., cuboid, cube or prism) and arrangements (at the same height as the transformer tank 12, further above, etc.) of the oil expansion tank 20 are conceivable.

Oil 40 is contained in the flow channel 30 and the housing 26 of the oil expansion tank 20. The oil expansion tank 20 is used to accommodate oil because of thermal expansion of the oil when temperature fluctuations occur in the transformer 15, caused by load changes or changes in the ambient temperature. As indicated by the surface 42 of the oil in the oil expansion tank 20, the oil level changes accordingly in the oil expansion tank 20. The oil 40 in the oil expansion tank 20 compresses a membrane 28 depending on the oil level 42 in the oil expansion tank 20, which is decompressed again when the oil level 42 drops. A second opening 24 in an upper end of the oil expansion tank 20 is fitted with a pressure relief valve 50 for releasing excess gas. The oil transformer further comprises an oil level measuring device 25 for measuring the oil level 42 in the oil expansion tank 20. Other components of the oil transformer, such as the transformer core, the coils and a Buchholz protection relay, are not shown in the schematic representation of FIG. 1 for clarity. As an alternative to the embodiment of the oil expansion tank 20 with the membrane 28, an oil expansion tank of the Atmoseal type or another type can also be provided.

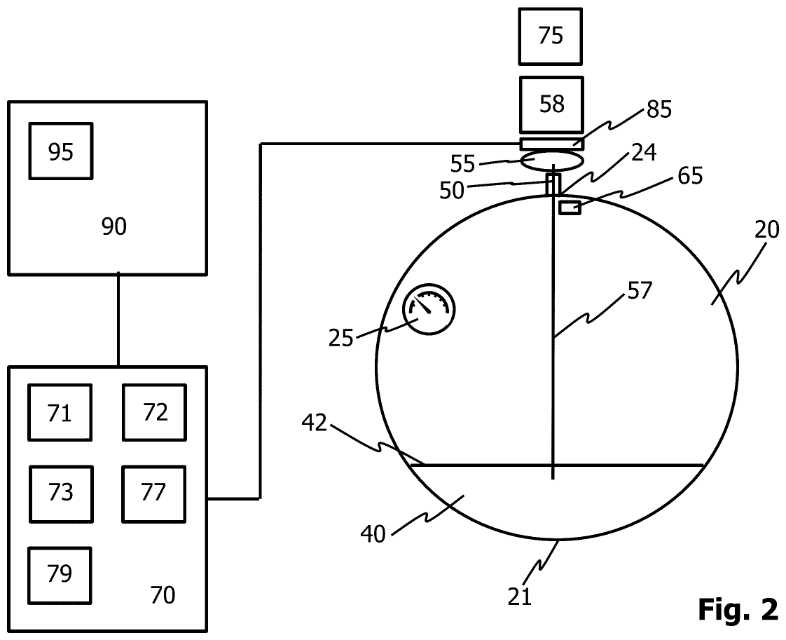

FIG. 2 shows a schematic representation of an exemplary embodiment of an oil expansion tank with a device for determining the oil quality. The oil expansion tank 20 is the oil expansion tank 20 shown in FIG. 1, wherein identical reference numerals in FIGS. 1 and 2 refer to the same elements. The arrangement shown in FIG. 2 comprises a cell 55 for receiving oil 40 from the oil expansion tank 20, means 57, 58 for conveying oil 40 from the oil expansion tank 20 into cell 55, an antenna 85 for applying the oil 40 in cell 55 with an electromagnetic signal, a sensor 70 electrically connected to antenna 85 for measuring an oil quality of the oil transformer 10, and a processing device 90.

The means 57, 58 for conveying oil 40 from the oil expansion tank 20 into cell 55 comprise a hose 57, at least partially arranged in the oil expansion tank 20, with a first end that extends into the oil 40 in the oil expansion tank 20, and a second end that is connected to cell 55, as well as a pump 58 for pumping the oil 40 from the oil expansion tank 20 into cell 55. Hose 57 extends through opening 24, with cell 55 being located outside oil expansion tank 20 at opening 24. The opening can also be a vent opening on oil expansion tank 20.

In FIG. 2, a minimum oil level 42 in oil expansion tank 20 is shown, with hose 57 designed so that the first end of hose 57 always extends below the minimum oil level 42. Pump 58 pumps oil 40 from oil expansion tank 20 into cell 55. It is also conceivable for pump 58 to pump oil 40 through cell 55, i.e., the oil 40 is returned to oil expansion tank 20. Pump 58 is designed as an electric pump and is electrically connected to antenna 85. Pump 58 is located close to antenna 85.

According to an alternative embodiment (not shown), cell 55 is located in oil expansion tank 20, in particular above a maximum oil filling level of oil expansion tank 20. antenna 85 is mounted on an outer wall of cell 55 and electrically connected to sensor 70.

Opposite the underside 21 of the oil expansion tank 20, there is an opening 24 at an upper end of the oil expansion tank 20, sealed by a pressure relief valve 50. Above opening 24, antenna 85 is arranged, which is electrically connected to sensor 70.

Sensor 70 comprises a baseband transmitter 71, a baseband receiver 72 and a digital backend 73, and is electrically connected via a cable to processing device 90. Baseband transmitter 71 generates pulsed excitation signals for antenna 85, with reflected/propagated signals being forwarded to baseband receiver 72. Digital backend 73 also includes semiconductor memory units, random access memory (RAM) units and trusted platform modules (TPMs) that meet necessary security and cryptographic requirements. TPM modules also help secure hardware with integrated cryptographic keys and provide mechanisms for user authentication and authorization. In addition, TPM modules are used to sign the sensor raw data in order to make the data authentic and feed it into blockchain technologies. Digital backend 73 controls the transmission and reception of the signals by baseband transmitter 71 and baseband receiver 72. In this process, sensor 70 is configured to transmit and receive pulsed signals at a frequency of 1 Hz to 3000 GHz via antenna 85. Preferably, the system operates in the ultra-wideband range, so that sensor 70 analyzes the signal in the wide frequency range from 0.1 GHz to 6.0 GHz, and antenna 85 transmits and receives signals in the range from 300 MHz to 300 GHz. Sensor 70 transmits and receives measurement signals via antenna 85, with which the quality of the oil in oil expansion tank 20 can be determined. Antenna 85 is a complementary split-ring resonator or a planar resonance-based sensor electrode. Other types of electrodes/probes that are designed to optimize parasitic effects and double-layer effects with the oil can also be used. Furthermore, sensor 70 includes a communication device 79 for communication with processing device 90. Communication device 79 can comprise wireless interfaces (for example LTE-M or NB-IoT cellular connection SoC modules, WiFi, BLE or NFC interfaces) and/or wired interfaces (for example I2C, SPI, UART, ADC, PWM, HDMI, VGA, ETHERNET interface).

The signals generated in baseband transmitter 71 of sensor 70 can be produced using cost-effective semiconductor flip-flops and shift registers. Depending on the level of detail and resolution of the oil analysis required, the frequency of the input signal can be varied by changing the configurations of the flip-flops and shift registers.

Processing device 90 is a laptop computer or any other type of computer that is configured to process and visualize the measured values acquired by sensor 70. Processing device 90 can also comprise microcontroller units, graphics processing units (GPU) and/or central processing units (CPU) or neural processing units (NPU) in order to perform necessary calculations for running machine learning with machine learning module 95. In particular, processing device 90 is configured to calculate values relating to color, water content and/or acid value of the oil based on the measured values acquired by sensor 70. Processing device 90 can also calculate other values mentioned above with respect to the physical property and the chemical property. For example, a fast Fourier transform can be applied to the measurement data.

The oil transformer also includes a temperature, gas and/or vibration sensor 65. The temperature, gas and/or vibration sensor 65 is located in oil expansion tank 20. The temperature, gas and/or vibration sensor 65 can also be arranged outside or at the connection interface of opening 24. The temperature, gas and/or vibration sensor 65 is configured to send measurement data to sensor 70 and/or to processing device 90. For this purpose, the temperature, gas and/or vibration sensor 65 can include a communication interface that enables communication with sensor 70 and/or processing device 90. The communication interface can be arranged at opening 24 and, for example, provide a wired connection to sensor 70. If the temperature, gas and/or vibration sensor 65 is designed as a gas sensor, it can be configured to detect abnormalities in the gas generated by the oil transformer. The sensor can also include additional sensor and electronic components that monitor a regular operation and a condition of the oil transformer.

For a precise determination of the oil quality of the oil in oil transformer 10, processing device 90 is configured to compare the electromagnetic signals received by the sensor in the time domain and/or frequency domain with first and second signals in the time domain and/or frequency domain. The first signals in the time domain and/or frequency domain are classified with respect to a physical property of oil, and the second signals in the time domain and/or frequency domain are classified with respect to a chemical property of oil. The physical property is one of viscosity, flash point, interfacial tension, color and density, and classification with respect to the physical property of oil concerns an oil quality. The chemical property is one of acid value, chemical composition, in particular changes in the chemical composition that arise through involvement of dissolved gases, moisture and the influence of paper polymerization, and classification with respect to the chemical property of oil concerns an oil quality. Based on the comparisons, processing device 90 determines the oil quality of oil 40 in oil transformer 10.

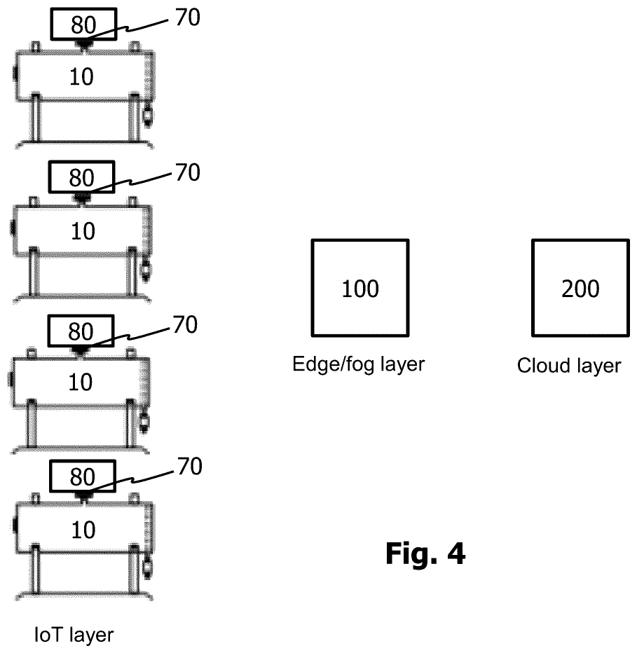

The comparison between the signals and the received signal can be carried out using a machine learning model, with the model having been trained on the predefined signals to find weights and parameters. The machine learning model then predicts the chemical or physical property of the oil and either provides a value or a probability distribution. The machine learning processing can take place in machine learning module 95, in processing device 90 or in cloud platform 200 (see FIG. 4). The described machine learning method also solves the inverse problem by determining the physical and chemical factors that influence the overall condition of the oil transformer.

The comparisons can be carried out by separate machine learning models, for example one for the first signals to predict the color and one for the second signal to predict the moisture content. Alternatively, the machine learning model can generate predictions for one or more chemical and physical properties in a single computational process.

For example, the first signals in the time domain and/or frequency domain are classified with respect to a color of oil. Thus, it can be taken into account that if the oil's color darkens from pale yellow to yellow, to light yellow, to amber, to brown, to dark brown and to black, the oil quality successively deteriorates. For example, the second signals in the time domain and/or frequency domain are classified with respect to moisture in oil.

Processing device 90 is involved in preprocessing the data, i.e. in the process of translating/deriving the electromagnetic signal into a physical value (for example, converting the Fourier transform output of the received electromagnetic signal into the moisture content of the oil) and excluding unwanted data packets from the original signal.

For the comparisons performed in the processing device, amplitude values (e.g., in dB) over time (e.g., in ms) can be compared with predefined and classified amplitude values (e.g., in dB) over time (e.g., in ms). In addition, a power spectrum calculated by means of a fast Fourier transform can be used, i.e., amplitude values (e.g., in dB) over frequency (e.g., in GHz) can be compared with predefined and classified amplitude values (e.g., in dB) over frequency (e.g., in GHz). The comparisons can be carried out using correlations (digital signal processing) or any type of output in different scales (e.g., logarithmic scale) and/or different graphical representations (e.g., Nyquist plot, Bode plot, etc.) in order to compare changes in phase angle and amplitude.

Optionally, processing device 90 can be configured to compare the received signals in the time domain and/or frequency domain with third and fourth signals in the time domain and/or frequency domain. The third signals in the time domain and/or frequency domain are classified with respect to a physical property of oil that differs from the classified physical property of oil of the first signals in the time domain and/or frequency domain, and the fourth signals in the time domain and/or frequency domain are classified with respect to a chemical property of oil that differs from the classified chemical property of oil of the second signals in the time domain and/or frequency domain. As with the first signals, the physical property in the third signals can be one of viscosity, flash point, interfacial tension, color and density, and classification with respect to the physical property of oil concerns an oil quality. Correspondingly, as with the second signals, the chemical property in the fourth signals can be one of acid value, chemical composition, in particular changes in the chemical composition that arise through involvement of dissolved gases, moisture and the influence of paper polymerization, and classification with respect to the chemical property of oil concerns an oil quality.

For example, the third signals in the time domain and/or frequency domain are classified with respect to a viscosity of oil, and the fourth signals in the time domain and/or frequency domain are classified with respect to an acid value of oil.

Processing device 90 is also configured to determine, based on the transmitted and received signals, an impedance, a conductance, an admittance and/or a susceptance in the time domain and/or frequency domain, and to compare the impedance, the conductance, the admittance and/or the susceptance in the time domain and/or frequency domain with the first and second signals in the time domain and/or frequency domain. Additionally, processing device 90 can compare the impedance, the conductance, the admittance and/or the susceptance in the time domain and/or frequency domain with the third and fourth signals in the time domain and/or frequency domain. The above-mentioned values can also be arbitrarily combined and compared with each other.

Moreover, processing device 90 is able to analyze historical data recorded by the device and/or other installed similar devices. Recorded data stored in the cloud can be used to find patterns and structures in the data in the form of an unsupervised machine learning module. Training of the machine learning module takes place in processing device 90 or another laptop/computer connected to the cloud.

The patterns and structures found in the data are used for qualitative and quantitative monitoring of the oil transformer 10 and can be further utilized in applications such as lifetime prediction of the oil transformer, prediction of transformer performance and capacity detection based on oil quality, active load management based on transformer capacity and transformer recommendations. The calculations of the patterns and the structure take place in processing device 90 or in cloud platform 200 (see FIG. 4).

As can be seen from FIG. 2, processing device 90 also comprises a machine learning module 95, which is configured to optimize the comparisons in processing device 90 based on the classifications of the first, second, third and/or fourth signals. The first, second, third and/or fourth signals can in particular be predefined signals.

The comparison between the predefined signals and the received signal can be carried out by means of a machine learning model that has been trained on the predefined signals to find weights and parameters. The machine learning model then predicts the chemical or physical property of the oil and provides either a value or a probability distribution. The machine learning processing can take place in machine learning module 95, in processing device 90 or in cloud platform 200 (see FIG. 4).

The device shown in FIG. 2 also comprises a temperature stabilizer 75 that is configured to keep the temperature of the extracted oil of oil transformer 10 constant.

An automatic calibration device 77 is provided in the sensor, which is configured to remove irregularities, noise and/or attenuation in the received electromagnetic signals. The automatic calibration device 77 can also be provided in processing device 90.

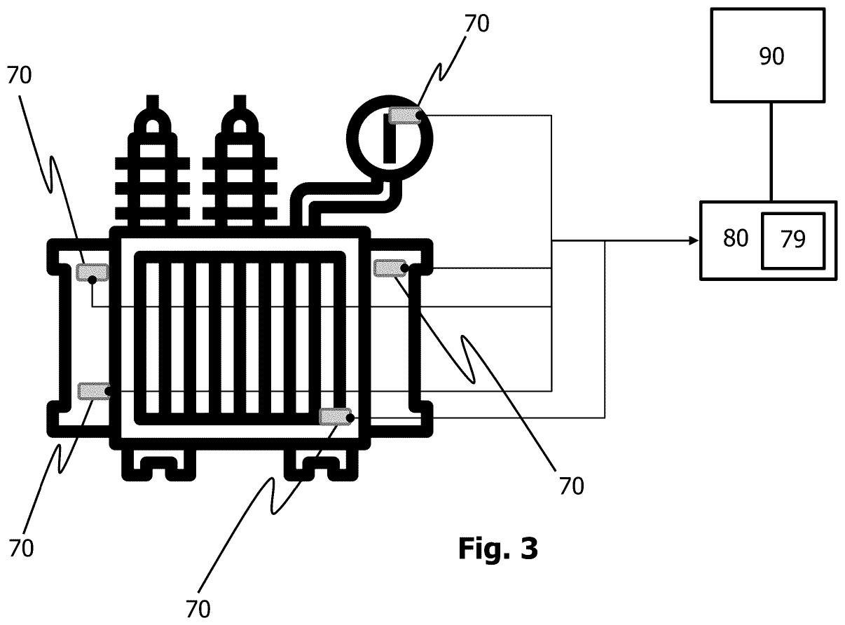

FIG. 3 shows a schematic representation of a second exemplary embodiment of an oil transformer with a device for determining the oil quality. The oil transformer can be oil transformer 10 shown in FIGS. 1 and 2 or another oil transformer (such as a hermetically sealed or any air-permeable oil transformer used in distribution and transmission networks). Identical reference numerals refer to the same elements, so no repeated explanation is provided.

The oil transformer comprises a plurality of openings. In each of the openings, there is a sensor 70. Each sensor 70 sends the received electromagnetic signals to an aggregation device 80, which aggregates the signals. Aggregation device 80 comprises a communication device 79. Communication device 79 sends the aggregated signals to processing device 90 or a cloud platform (not shown in FIG. 3). The transmission can take place at the same time, in particular in real time.

According to an alternative embodiment, only one sensor 70 is used, and sensor 70 is brought in succession to each of the openings of the oil transformer in order to measure the oil quality in the oil transformer. The received electromagnetic signals can be temporarily stored in a storage device in sensor 70 and then aggregated by aggregation device 80.

In summary, the exemplary embodiment of FIG. 3 can be described as follows: Sensors 70 of oil transformer 10 are connected to aggregation device 80 via a wireless or wired communication link. The data/signals received by sensors 70 can be processed with the aid of a machine learning module, see for example machine learning module 95 in FIG. 2, and then sent to aggregation device 80. Sensors 70 can also communicate directly or via the edge/fog layer with a cloud platform (not shown in FIG. 3, see, however, the edge or fog layer with the highly secure gateway 100 in FIG. 4) by means of communication device 79. Communication device 79 is configured to communicate as described with respect to communication device 79 in FIG. 2. Aggregation device 80 communicates wirelessly with a hypersecure gateway that uses an edge or fog layer (not shown in FIG. 3, see the edge or fog layer with hypersecure gateway 100 in FIG. 4). With the data collected in aggregation device 80, sensor fusion algorithms can be applied to study a real-time behavior of the oil transformer.

FIG. 4 shows a schematic representation of a third exemplary embodiment of a system for a plurality of oil transformers, with four oil transformers shown by way of example. The oil transformers can each be oil transformer 10 shown in FIG. 1 or 3 or another oil transformer, and sensor 70 can each be sensor 70 shown in FIG. 2 or 3 or another sensor. Aggregation device 80 can be the aggregation device 80 shown in FIG. 3. Identical reference numerals refer to the same elements, so no repeated explanation is given.

The oil transformers 10 are part of an electric power supply network (not shown in FIG. 4). Sensors 70 are configured to send the received or the aggregated received data to a cloud platform 200 in the cloud layer via an edge or fog layer that uses a hypersecure gateway 100. The received data is then processed there.

A processing device 90 (not shown in FIG. 4) can be provided in each sensor 70 and in each aggregation device 80. Aggregation device 80 is part of the Internet of Things (IOT) layer and communicates wirelessly with the edge/fog layer. The cloud layer runs various applications, such as making lifetime predictions for components of the oil-containing electrical power device or transmission device (10) based on the determined oil quality, detecting anomalies of the oil-containing electrical power device or transmission device (10), making predictions of transformer performance and capacity detection of the oil-containing electrical power device or transmission device (10), performing active load management based on a transformer capacity of the oil-containing electrical power device or transmission device (10), and/or making transformer recommendations for the oil-containing electrical power device or transmission device (10) from a plurality of transformers. The hypersecure gateway 100 is provided for secure communication between the cloud layer and the plurality of sensors 70 or the plurality of aggregation devices 80 in the IoT layer. A multi-layer architecture is therefore considered, comprising an IoT layer, an edge/fog layer and a cloud layer, in order to describe a decentralized data processing structure that lies between the cloud and the devices that generate data. This flexible architecture allows users to place resources, including applications and the data they generate, in logical locations to improve performance. For this purpose, sensors 70 are located in the IoT layer, hypersecure gateway 100 in the edge/fog layer, and measurement data processing is performed in the cloud layer 200.

Sensors 70 function in the IoT layer as an IoT awareness layer in a smart grid. The edge/fog layer enables secure communication between the IoT layer and the cloud layer. For this purpose, the edge/fog layer is configured to perform authorization, dual certificate authentication and data preprocessing for anomaly detection. In addition, a high availability and fail-safety of a network/transformer monitoring method is ensured. Moreover, dual virtualization is possible, since this layer is empowered by virtualization technology to migrate from connected environments to another one and to prevent cascading faulty data through the system. This is done by migrating functions and data from dedicated hardware that is at risk to other hardware. The cloud layer is used to provide applications for monitoring, historical data analysis, AI-based applications and visualizations.

For setting up secure network communication, either transmission control protocol (TCP) and/or user datagram protocol (UDP)-based protocols can be used for data transport from the physical IoT layer to the edge layer, and any TCP and/or internet protocol (IP)-based communication protocol can be used from the edge layer to the cloud layer.

A module for artificial intelligence (AI), e.g. machine learning module 95, can also be present in each of sensors 70, which can be used to optimize the data stored in aggregation device 80. Furthermore, AI and machine learning capabilities can be provided for other entities.

According to further developments of the processing devices 90 shown in FIGS. 2 and 3, the respective processing devices 90 can be configured, based on the determined oil quality, to make lifetime predictions for components of the oil-containing electrical power device or transmission device (10), to detect anomalies of the oil-containing electrical power device or transmission device (10), to make predictions of transformer performance and capacity detection of the oil-containing electrical power device or transmission device (10), to perform active load management based on a transformer capacity of the oil-containing electrical power device or transmission device (10), and/or to make transformer recommendations of the oil-containing electrical power device or transmission device (10) from a plurality of transformers.

One exemplary embodiment of a method for determining an oil quality of an oil transformer with the devices for determining the oil quality shown in FIGS. 2 to 4 comprises the method steps: arranging a plurality of sensors 70 of the device at or in different openings and/or valves of oil transformer 10, and transmitting, by the plurality of sensors 70, the electromagnetic signals into the oil in oil transformer 10.

Thus, a device, a system and a method for oil transformers are provided, with the aid of which the oil quality in an oil transformer can be determined in a simple and precise manner. As described above, the exemplary embodiments of FIGS. 1 to 4 can also be extended to oil-containing electrical power devices or transmission devices.

In the presented examples, different features and functions of the present disclosure have been described separately from each other as well as in certain combinations. However, it will be understood that many of these features and functions, where not explicitly excluded, are freely combinable with each other.

Thus, both oil transformers with an oil expansion tank and oil transformers without an oil expansion tank can be used. The comparisons of the received electromagnetic signals with the signals that indicate properties of the oil can take place in one of the sensors 70, in processing device 90, in a central unit, or in a cloud server.

1. A device for an oil-containing electrical power device or transmission device, comprising:
a sensor that is configured to transmit one or more electromagnetic signals with variable frequencies in the range from 1 Hz to 3000 GHz, at the same time into oil in the oil-containing electrical power device or transmission device and to receive reflected and/or propagated electromagnetic signals, and
a processing device configured to compare the received signals in a time domain and/or a frequency domain with first and second signals in the time domain and/or the frequency domain, wherein:
the first signals in the time domain and/or the frequency domain are classified with respect to a physical property of oil, and
the second signals in the time domain and/or the frequency domain are classified with respect to a chemical property of oil, and
wherein the processing device is further configured to determine, based on the comparisons, an oil quality of the oil in the oil-containing electrical power device or transmission device.

2. The device according to claim 1, wherein:
the physical property is one of viscosity, flash point, interfacial tension, color and density,
the classification with respect to the physical property of oil concerns the oil quality,
the chemical property is one of acid value, chemical composition, and
the classification with respect to the chemical property of oil concerns an oil the oil quality.

3. The device according to claim 1, wherein the processing device is configured to compare the received signals in the time domain and/or the frequency domain with third and fourth signals in the time domain and/or the frequency domain, wherein:
the third signals in the time domain and/or the frequency domain are classified with respect to a physical property of oil that differs from the classified physical property of oil of the first signals in the time domain and/or the frequency domain, and
the fourth signals in the time domain and/or the frequency domain are classified with respect to a chemical property of oil that differs from the classified chemical property of oil of the second signals in the time domain and/or the frequency domain.

4. The device according to claim 1, wherein the processing device is configured to determine, based on the transmitted and received signals, an impedance, a conductance, an admittance, a susceptance and/or combinations thereof in the time domain and/or the frequency domain and to compare the impedance, the conductance, the admittance, the susceptance and/or the combinations thereof in the time domain and/or the frequency domain with the first and second signals in the time domain and/or frequency the frequency domain.

5. The device according to claim 4, further comprising machine learning models that are configured to be processed on a machine learning module and to determine, based on the impedance, the conductance, the admittance, the susceptance and/or the combinations thereof, values relating to the physical property and the chemical property of the oil.

6. The device according to claim 1, further comprising a machine learning module that is configured to optimize the comparisons in the processing device based on the classifications of the first and the second signals, with the machine learning module using the first and second signals as training data.

7. The device according to claim 1, wherein the sensor is configured to transmit and receive the electromagnetic signals at or in different openings of the oil-containing electrical power device or transmission device.

8. The device according to claim 1, further comprising a plurality of sensors at or in different openings of the oil-containing electrical power device or transmission device, wherein the plurality of sensors is configured to transmit one or more electromagnetic signals with variable frequencies in the range from 1 Hz to 3000 GHz, at the same time into the oil, to receive reflected and/or propagated electromagnetic signals, and to process the received signals.

9. The device according to claim 8, further comprising an aggregation device with a communication device that is configured to receive the processed signals from the plurality of sensors, wherein:
the aggregation device is configured to aggregate the processed signals from the plurality of sensors, and
the communication device is configured to transmit the aggregated signals.

10. The device according to claim 1, wherein the processing device is configured to process the received signals at the same time.

11. The device according to claim 1, further comprising a temperature stabilizer that is configured to keep the temperature of the oil constant while transmitting and receiving the electromagnetic signals.

12. The device according to claim 1, further comprising an automated and controllable calibration device that is configured to remove irregularities in the received electromagnetic signals.

13. The device according to claim 1, further comprising an antenna that is configured to transmit the electromagnetic signals of the sensor into the oil and to receive the reflected and/or propagated electromagnetic signals, wherein the antenna comprises a complementary split-ring resonator, planar resonance-based sensor electrodes or electrodes/probes that are designed to optimize parasitic effects and double-layer effects of the oil.

14. The device according to claim 1, further comprising a cloud platform configured to:
make, based on the determined oil quality, lifetime predictions for components of the oil-containing electrical power device or transmission device,
detect anomalies of the oil-containing electrical power device or transmission device,
make predictions of transformer performance and capacity detection of the oil-containing electrical power device or transmission device,
perform active load management based on a transformer capacity of the oil-containing electrical power device or transmission device, and/or
make transformer recommendations for the oil-containing electrical power device or transmission device from a plurality of oil transformers.

15. A system, comprising:
one or a plurality of oil-containing electrical power devices or transmission devices, and
for each of the one or the plurality of oil-containing electrical power devices or transmission devices, a device comprising:
a sensor that is configured to transmit one or more electromagnetic signals with variable frequencies in the range from 1 Hz to 3000 GHz at the same time into oil in the oil-containing electrical power device or transmission device and to receive reflected and/or propagated electromagnetic signals, and
a processing device configured to:
compare the received signals in a time domain and/or a frequency domain with first and second signals in the time domain and/or the frequency domain, wherein:
the first signals in the time domain and/or the frequency domain are classified with respect to a physical property of oil, and
the second signals in the time domain and/or the frequency domain are classified with respect to a chemical property of oil; and
determine, based on the comparisons, an oil quality of the oil in the oil-containing electrical power device or transmission device.

16. A method for determining an oil quality of an oil-containing electrical power device or transmission device, the method comprising:
arranging a plurality of sensors at or in different openings and/or valves of an oil-containing electrical power device or transmission device;
transmitting, by the plurality of sensors, electromagnetic signals with variable frequencies in the range from 1 Hz to 3000 GHz at the same time into the oil in the oil-containing electrical power device or transmission device;
receiving, by the plurality of sensors, reflected and/or propagated electromagnetic signals;
comparing, with a processing device, the received signals in a time domain and/or a frequency domain with first and second signals in the time domain and/or the frequency domain, wherein:
the first signals in the time domain and/or the frequency domain are classified with respect to a physical property of oil, and
the second signals in the time domain and/or the frequency domain are classified with respect to a chemical property of oil; and
determining, with the processing device and based on the comparisons, an oil quality of the pol in the oil-containing electrical power device or transmission device.
